(19) United States
(12) Patent Application Publication
(10) Pub. No.: US 2005/0287573 A1
(43) Pub. Date: Dec. 29, 2005

(54) Lined multi-well plates

(75) Inventors: Shane J. Stafslien, Fargo, ND (US); James Allen Bahr, West Fargo, ND (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: North Dakota State University, Fargo, ND (US)

(21) Appl. No.: 11/155,313

(22) Filed: Jun. 17, 2005

Related U.S. Application Data
(60) Provisional application No. 60/581,023, filed on Jun. 18, 2004.

(51) Int. Cl. C12Q 1/68; C12M 1/34

(52) U.S. Cl. 435/6; 435/287.2

(57) ABSTRACT

A multi-well plate includes a plurality of wells, and each well has a base and one or more side walls. A liner is located adjacent the base of at least one of the plurality of wells. A method for forming a multi-well plate system includes providing a multi-well plate and applying a liner to a well of the multi-well plate.

### CROSS-REFERENCE TO RELATED APPLICATION(S)

The present application claims priority to U.S. Provisional Patent App. Ser. No. 60/581,023, entitled “Modified (Glass-Lined) Multi-Well Plates” by Shane J. Stafslien and James Allen Bahr, filed Jun. 18, 2004, which is hereby incorporated by reference in its entirety.

### STATEMENT OF GOVERNMENT INTEREST

The present invention was made, in part, with government finding under the Office of Naval Research (ONR), Grant Nos. N00014-02-1-0794, N00014-03-1-0702 and N00014-04-1-0597. The U.S. Government has certain rights in this invention.

### BACKGROUND OF THE INVENTION

Materials are needed that exhibit antifouling and or easy release properties, for such applications as coatings for naval vessels. It is preferable to use a combinatorial high-throughput workflow when developing such materials. In such combinatorial, high-throughput methods, a need has emerged in the realm of screening and successfully identifying promising candidates from the numerous amounts of materials generated. This need mandates that all valid screening protocols and procedures are rapid, efficient and economically feasible.

A biological assay is currently being developed and implemented as one such screening protocol to assess the antifouling/foul-release properties of novel coating materials. Coatings are challenged with various marine bacteria and are assessed by their ability to inhibit and or remove bacterial films (biofilms). In this regard, a multiwell plate format amenable to high-throughput methodologies is utilized for the parallel assessment of coatings. However, commercially available formats do not adequately fit the need required for this assay.

Commercially available plates fabricated from materials such as polystyrene or polycarbonate are not amenable to common coating solvents such as MEK, toluene, acetone etc. Solvents such as these attack the integrity of the plate facilitating a chemical reaction with the plate. This chemical reaction inhibits the formation of a suitable film needed for screening purposes.

Commercially available multiwell plates fabricated from materials such as glass or polypropylene permit the deposition of common coating solvents. However, these plates are not amenable to high-throughput workflow because the plates are non-disposable, high in cost (ranging in price as high as $400.00-$500.00 per plate) and have the potential to promote delamination of cured coating materials from the wells upon exposure to aqueous environments. Given their high price, glass or polypropylene multiwell plates would require re-use.

To be able to re-use the plates, the plates would need to be washed or have the coating removed using some similar method. Given that the coatings often cure and harden, removal of the coating from the glass or polypropelyne plate can be difficult. Further, the removal process can etch the glass, or otherwise damage or affect the surface of the plate. Etched or otherwise damaged plates have a negative effect on the high throughput workflow because it may be difficult to apply the coatings as desired to the plates.

SensoPlate™ glass bottom, black polystyrene, multi well plates are available for purchase from Bellco Glass, Inc. of Vineland, N.J. SensoPlates™ are composed of a high quality optical glass bonded to low auto-fluorescence black polystyrene. The intended applications for this product include high-resolution imaging, sensitive fluorescence and confocal microscopy applications. Though this format could be employed for other high-throughput workflow, several drawbacks or limitations limit its usage.

SensoPlates™ are expensive (with prices ranging from $39.05 to $222.80 a plate). Once again, the cost of these plates would require their re-use, and the plates are not disposable. The glass bottom is bonded to the polystyrene top. Organic or other common coatings solvents could potentially attack this bond and compromise the integrity of the plate. Depending on the application, other types of material may be advantageous for use as the bottom of each well, (such as metal or plastics, etc.). In this regard, the SensoPlates™ would require further modifications to fit this need.

### BRIEF SUMMARY OF THE INVENTION

The present invention comprises a modified multi-well plate, and a method of forming a modified multi-well plate. The multi-well plate comprises a standard multi-well plate, with a non-reactive liner. The inventive plate enables the deposition of a variety of coating formulations (containing common coating solvents) while substantially limiting the solvent interaction with the plate material. Thus, the non-reactive liner minimizes the affect of the solvent on the wells, ensuring abetter deposition, and better test results.

### BRIEF DESCRIPTION OF THE DRAWINGS

FIG. 1 is a perspective view of a polystyrene multi-well plate in which a coating formulation was deposited in the first two rows.

FIG. 2 is a perspective view of a multi-well plate having an adhesive applied in the bottom of each well.

FIG. 3 is a top view of one suitable liner for use with the present invention.

FIG. 4A is a perspective view of the modified multi-well plate of the present invention.

FIG. 4B is a side cross sectional view of the modified multi-well plate of the present invention.

FIG. 5 is a perspective view of an extraction template.

FIG. 6 is a bottom view of a block.

FIG. 7 is an exploded cross-sectional view of a portion of the extraction template of FIG. 5 and a portion of a multi-well plate.

FIG. 8 is a side perspective view of the extraction template and the multi-well plate in a clamping device.

FIG. 9 is a cross-sectional view of a portion of the extraction template 40 mounted to a portion of the multi-well plate 20.

FIGS. 10-12 are graphs of optical density for tests using the present invention.

FIG. 13 is a graph of static contact angle measurements and surface free energy calculations obtained used in the present invention.

### DETAILED DESCRIPTION

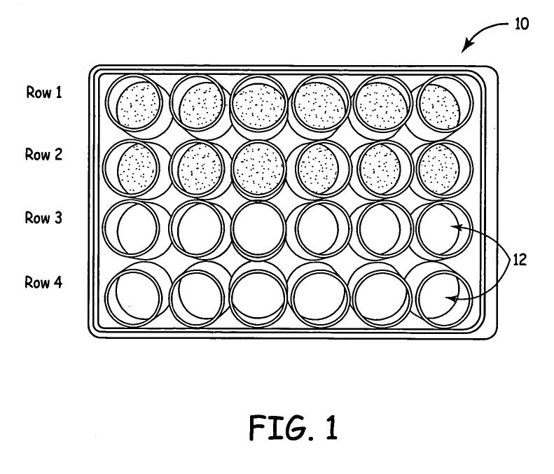

FIG. 1 is a perspective view of a commercially available polystyrene multi-well plate 10. The multi-well plate 10comprises twenty-four wells 12 arranged in four rows. The wells 12 of the first and second rows were deposited with a coating formulation diluted in MEK. The coating/MEK mixture reacted with the wells 12 of the plate 10 upon curing at room temperature. Due to this reaction, the wells 12 of the first and second rows have a cloudy, discolored appearance, while the wells 12 in the third and fourth rows remain clear.

The multi-well plate 10 is commonly used in performing testing or screenings of a wide variety of materials. Specifically, when performing tests on or screenings of various coatings, the multi-well plates may be exposed to a variety of solvents, such as organic solvents, or coatings that contain solvents. The solvent may react with the plate 10 in such a way that adversely affects the use of the plate 10 in the coating screening or test. For instance, the solvent applied to or present in the coating may react with the wells 12 of the plate 10. Such a reaction adversely affects the ability to obtain a uniform coating on the bottom of the wells 12, as is desired during high throughput screening of multiple variations of coatings. Such a reaction also contaminates the samples held in the wells 12, and may otherwise adversely affect the samples or tests performed on the samples.

The present invention addresses these concerns by modifying the multi-well plate by adding a non-reactive liner to each well 12. Adding a non-reactive liner to the bottom of each well 12 limits the reaction between the coating in the well 12 and well 12. As a result, a better, more uniform coating is achieved in the wells 12.

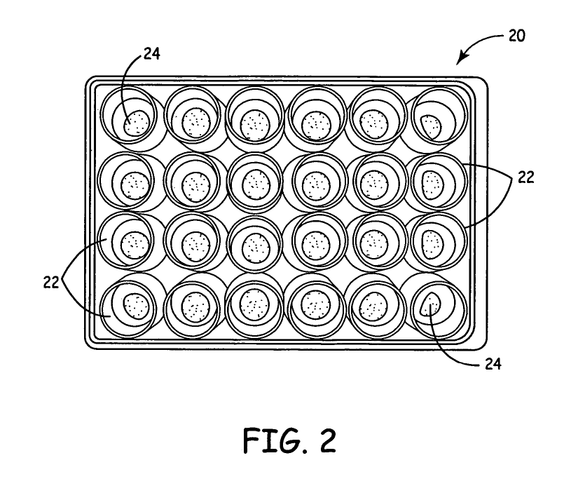

The method of forming the modified multi-well plate is illustrated in FIGS. 2-4A. FIG. 2 is a perspective view of a multi-well plate 20 having twenty-four wells 22 suitable for use with the present invention. To form the modified multi-well plate of the present invention, an amount of adhesive 24 is applied to the bottom of each well 22.

The adhesive 24 may comprise any suitable material, such as glue, epoxy, vacuum grease or the like, capable of holding a liner in the well. Further, the adhesive 24 is preferably chosen to be compatible with the coating or other material to be held in the wells 22. The adhesive 24 may likewise be chosen so that it is compatible with any tests performed using the multi-well plate 20.

For instance, if optical tests will be used in connection with the plate 20, the adhesive 24 chosen may have the desired optical qualities, such as low auto-fluorescence at various wavelengths of light. In other situations, it maybe desirable to have an optically transparent adhesive layer 24.

The adhesive 24 maybe a low-viscosity adhesive suitable for application using a pipette. More preferably, the adhesive 24 is a low viscosity adhesive suitable for application using multiple pipetting performed by a robot to allow for mass production of the plates 20. The adhesive 24 may have a long pot life, so as not to cure before being covered with a liner. A pot life of 30 minutes or longer is preferred. In addition, thermal curing below 100° C. is preferred.

One suitable type of adhesive is a silicone two-part coating that is capable of curing within 15 minutes. The silicone based adhesive maybe diluted with a solvent to obtain the desired viscosity. It is desired that the adhesive 24 be viscous enough to be easily spread when a liner is applied, so that the adhesive covers about the entire bottom surface of the wells 22.

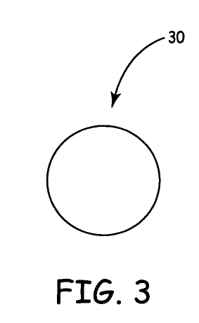

FIG. 3 is a top view of a liner 30 suitable for use with the present invention. As shown in the example in FIG. 3, the liner 30 is circular with a diameter of about 15 mm. The liner 30 maybe formed of any suitable material that will exhibit the desired properties when the multi-well plate is put to use. For instance, suitable materials for the liner 30 may include glass, aluminum, titanium, stainless steel, PVC, polycarbonate, polyetherimide, polyetheretherketone (PEEK), polyimide, polytetraluoroethylene (PTFE), polyethylene, polypropylene, polyurethane, acetate, polyester, nylon, or other materials. If it is desired that the liner 30 be non-reactive with certain solvents or coatings containing solvents, the liner 30 maybe formed of glass. Glass liners 30 are also desirable in many applications because they form a flat surface, desirable for applications of coatings. When formed of glass, suitable sources for the liner 30 are commercially available cover slips for use with microscope analysis.

The liners 30 can be formed of materials selected based upon the desired application. The material used for the liner 30 can influence biological performance in testing applications. The liners 30 can be punched out of a larger sheet into a desired size and shape.

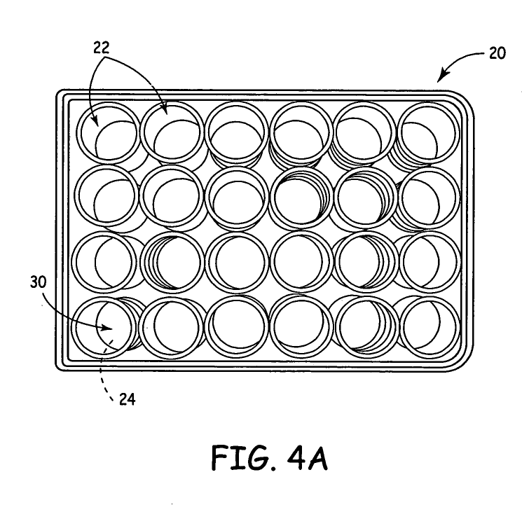

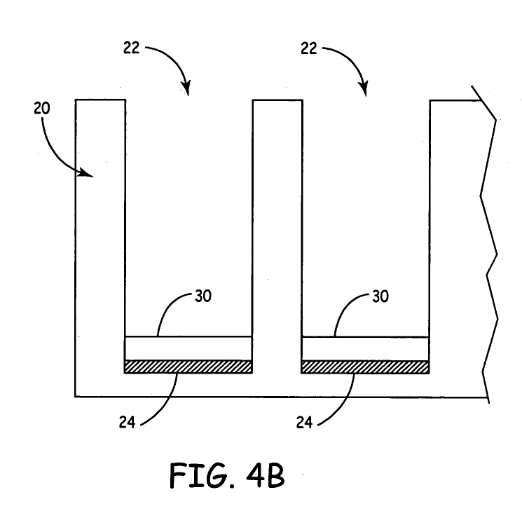

FIG. 4A is a perspective view of a modified multi-well plate 20 in which liners 30 have been applied to the adhesive 24 at the bottom of each well 22. Once the liners 30 are inserted, the adhesive 24 holds the liners 30 in place, such as by adhering, bonding, or otherwise attaching the liners 30 to the bottom of the wells 22.

FIG. 4B is a side cross-sectional view of the multi-well plate 20 illustrating the present invention. Shown in FIG. 4B is a portion of the multi-well plate 20 and two wells 22. Each well comprises a liner 30 and layer of adhesive 24. The layer of adhesive 24 is on the bottom surface of the well 22, and holds the liner 30 in the bottom of the well 22.

It will be recognized that multi-well plate 20 can be lined with liners 30 of different materials. For instance, some of the liners 30 can be glass and others formed of aluminum. Plates 20 lined with several types of liners 30 may be useful for biocompatibility studies.

The liners 30 are preferably sized to ensure a tight fit in the wells 22. Ensuring the liners 30 fit snugly into the wells 22 helps prevent coatings or solvents that are later applied to the wells 22 from seeping under the liner 30 and adversely reacting with the adhesive 24 used to hold the liner 30 in the well 22. Such a reaction may lead to contamination of the coating or other material applied to the well 22.

The intent of the liner 30 is to minimize the contact with any material other than the liner 30. However, some reaction between the coating material and the sides of the wells 22 may occur, and may even be preferable in some instances. For instance, the reaction between the coating and the sides of the wells 22 may serve to anchor the coating in the well 22, so that the coating does not delaminate during subsequent testing. At the same time, the reaction between the coating and the well is limited by the liner 30, so that the coating samples are less contaminated, and a better, more uniform and flat surface coating is achieved.

The amount of adhesive 24 applied to the bottom of the wells 22 will vary based on the desired strength of attachment between the liner 30 and the plate 20. The amount and viscosity of the adhesive is preferably such that when the liner 30 is added, the adhesive 24 easily flows over about the entire bottom surface of the well 22. If the amount of adhesive 24 is too little, or the adhesive 24 is not of the correct viscosity, the adhesive 24 will not cover the majority of the surface of the liner 30. In such instances, a coating or solvent applied to the well 22 may seep past the liner, and adversely interact with the adhesive 24, leading to contamination or other undesirable affects on the coating.

At the same time, the amount of adhesive 24 applied to the bottom of the wells 22 must not be so great that the adhesive 24 oozes out around the liner 30 when the liner is inserted. It has been found that about 25 micro liters is a suitable amount of adhesive.

The method of making the inventive multi-well plates comprises the following steps. Each well of a commercially available multi well plate, typically fabricated from non-glass material, has an appropriate amount of adhesive deposited on the bottom. The type of adhesive chosen depends on the desired properties of the finished multi-well plate. The amount of adhesive is an amount effective to adhere the desired liner to the bottom of each well.

The selected liner may be any suitable material, is preferably the same dimensions of each well, and is placed onto the adhesive drop. Pressure is applied to the liner to spread adhesive over the well bottom between the liner and the well bottom. The adhesive is then allowed to cure for an appropriate period of time. Upon curing, the liner is held in the bottom of the well.

Yet another method of liner attachment would be to soften the multi-well plate during manufacture, or otherwise cause the wells to expand slightly. Once the multi-well plate is softened, the liner is inserted and the multi-well plate is allowed to contract, shrinking the plate slightly around the liner inserts. This would provide an adhesive free mechanical attachment of the liner to the multi-well plate. If glass slides were installed in this fashion, the resulting plate would have optically transparent wells. Similarly, the inserts could also be inserted as part of the multi-well plate molding process to achieve an adhesive free mechanical bond.

The following advantages are provided via utilization of this modified format: First, the minimal interaction of coating material with base plate material facilitates a flat and smooth film surface on the anchored liner 30. Achieving a smooth, flat surface is imperative for accurate testing and screening of the coatings. For instance, in specific applications relating to antifouling and foul-release materials, a smooth flat surface is imperative for accurate analysis via optical imaging techniques.

Second, a minimal amount of coating-plate interaction may still take place around the perimeter of the well. This small amount of coating-plate interaction serves to “anchor” each coating securely to the bottom of the well. This has been demonstrated to inhibit the delamination of cured coating materials upon immersion in an aqueous environment such as salt water.

Third, the modified multi-well plates of the present invention are disposable, thereby eliminating the time intensive cleaning required for non-disposable formats. Fourth, the modified multiwell plate format is relatively low in cost. The modified multi-well plates maybe produced for less than about $5 per plate when mass produced. Such a low cost alternative is much more appropriate for high-throughput screening protocols that demand the utilization of numerous plates at one time.

The modified format could potentially handle a broad range of temperatures. In particular, the modified multi-well plate 20 is an improvement over other types of plates having a glass bottom affixed to polystyrene wells, such as the SensoPlate™. Specifically, the SenoPlates™ may suffer due to different thermal expansion coefficients between the glass bottom and polystyrene top. Because the glass is not continuous in the modified multi-well plastic, the liner 30 and plate 20 can adjust or expand as needed depending on the temperature at which the plastic is utilized. In contrast, the continuous “sheet” of glass bonded to the polystyrene top in the SensoPlate™ design may not be able to adjust or expand appropriately, thereby compromising the integrity of the plate.

Lastly, the modified multi-well plates are more durable than plates formed entirely or partially of glass. This increased durability allows for use of the modified multi-well plates in a variety of applications. For instance, the multi-well plates can be sent out in kit form, for off-site material deposition, and then returned to the lab for analysis.

Though disclosed as a twenty-four multi-well plate, the invention is not so limited, and may be useful for multi-well plates having fewer or greater wells. Similarly, though discussed in terms of using a polystyrene multi-well plate, the invention is not so limited. Other multi-well plates may benefit from the modification proposed in this invention. Further, though shown as a flat circular shape, the liner is not so limited. Other shapes of liners maybe more appropriate for various applications, for instance the liner may take the shape of a cup, and extend up the sides of the wells a small distance, or may take the shape of a cylinder, protecting the sides of the wells, but leaving the bottom unlined.

Applications of the modified multiwell plates described above could have potential and beneficial applications in the following areas, for example: analysis of antifouling materials; culturing of cell lines (i.e., Hepatocyte adhesion); screening of materials for use in coatings of artificial implants (i.e., HPA adhesion); any applications where the interaction of a chemical or biological solution, with a coating or polymeric sample is studied; etc.

An assay may generally be conducted as follows. Typically, coatings or other materials to be tested are first placed on liners 30 in wells 22 of the multi-well plate 20. Agents, biofilms or other materials can be added to the wells 22 in order to conduct an assay. At a desired point during an assay, materials can be extracted from the wells 22. However, biofilms or other materials may become attached and retained to the sides of wells 22 rather than only to the liners 30. In order to measure only materials on the liners 30, an apparatus and technique for extracting materials exclusively from the liners 30 can be used. More particularly, an extraction template according to the present invention can be used to withdraw materials substantially exclusively from the liners 30.

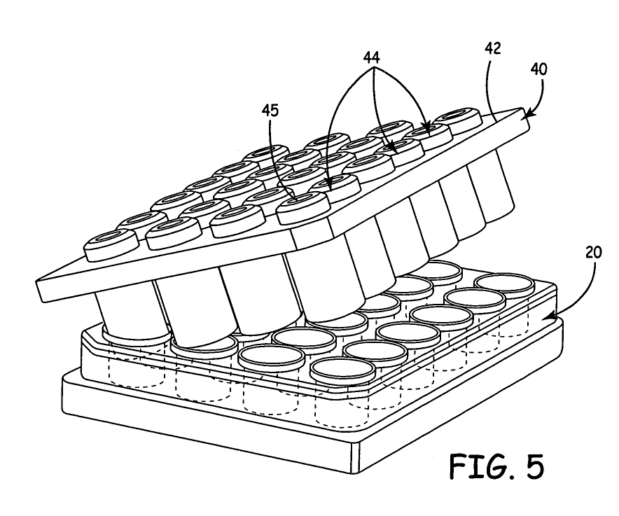

FIG. 5 is a perspective view of an extraction template 40, which includes a block 42 and one or more septa 44, positioned relative a multi-well pate 20 having wells 22. The septa 44 are generally formed of a chemically inert or resistant material, and have an opening 45a for a pipette tip and a skirt portion 45b.

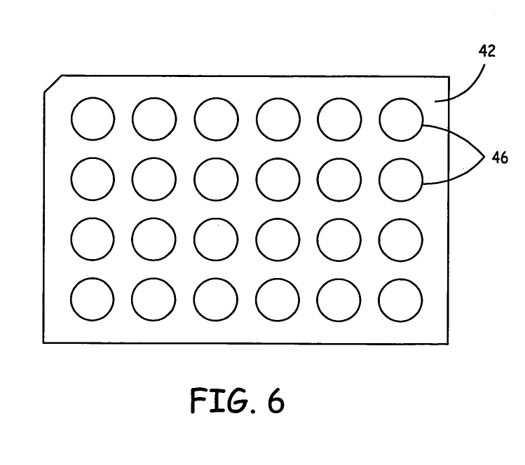

FIG. 6 is a bottom view of the block 42. The block 42 is generally formed to match the dimensions and configuration of a multi-well plate 20 with which it will be used. The block includes one or more holes 46, which are generally configured to correspond to the size, shape and configuration of the wells 22 of the multi-well plate 20. In one embodiment, the block 42 is 127 mm by 86 mm by 6.4 mm, the holes 46 are 13.4 mm in diameter, and the holes are spaced 19.4 mm from centers. The block 42 can be aluminum, or any other suitable material. The holes 46 can be machined in the block 42.

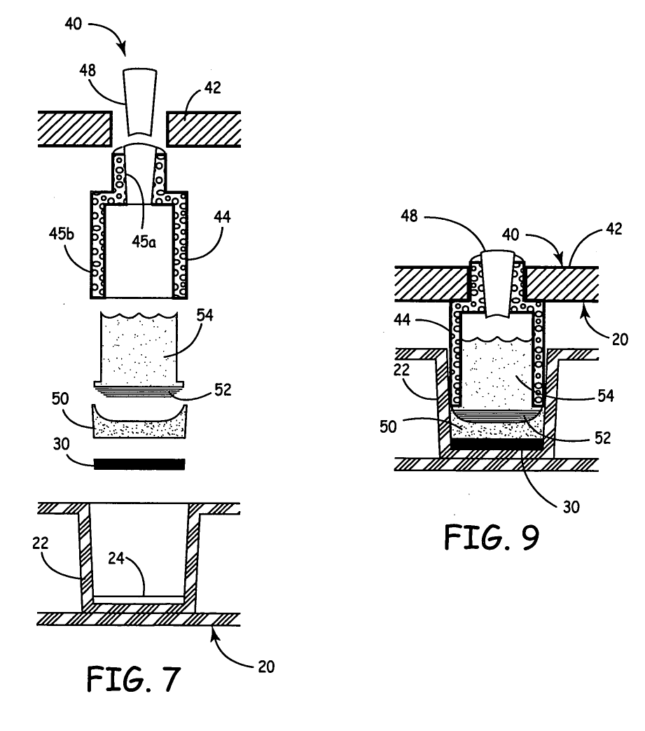

FIG. 7 is an exploded cross-sectional view of a portion of the extraction template 40 and a portion of a multi-well plate 20. The extraction template 40 includes septa 44 secured within the holes 46 of the block 42. An access tube or wedge clamp 48 is positioned within the opening 45 of the septa 44, for securing the septa 44 within the holes 46 by a press-fit. As shown in FIG. 7, a coating material 50 under analysis is positioned on the liner 30 of the multi-well plate 20. A biofilm 52, such as a crystal violet stained biofilm, is disposed on top of the coating material 50. An extraction solution 54, such as an acetic extraction solution, is located in the well 22 on top of the biofilm 52. Typically, the coating material 50 and the biofilm 52 have been previously prepared in the multi-well plate 20, and the extraction template 40 is then used to remove at the biofilm 52 exclusively from a surface of the coating material 50.

In order to mount the extraction template 40, the extraction template 40 is immersed in deionized water briefly and then tapped on a paper towel to remove any remaining water drops before being applied to the multi-well plate 20 containing the biofilm(s) 52. This step helps to lubricate the septa 44 and facilitate easy application into the wells 22. The extraction template 40 is positioned such that the septa 44 enter the wells 22 of the multi-well pate 20.

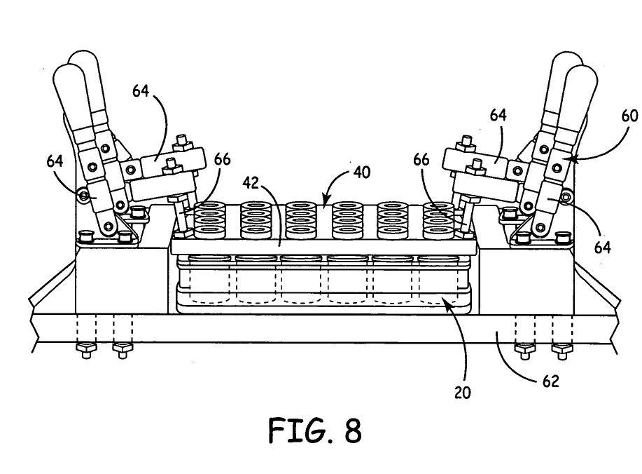

FIG. 8 is a side perspective view of the extraction template 40 and the multi-well pate 20 in a clamping device 60. The clamping device includes a base 62, one or more clamping levers 64, and one or more pressure applicators 66. In the embodiment shown in FIG. 8, there are four clamping levers 64 with four pressure applicators 66, each positioned relative to a corner of the block 42 of the template 40. Once the extraction template 40 has been applied to the multi-well plate 20, the plate 20 and template 40 are placed in the clamping device 60 to apply sufficient pressure to the template 40 so as to create a water tight seal at an interface between the coating material 50 and the septa 44. The pressure applicators 66 apply force in a generally downward direction to the block 42, which secures the multi-well plate 20 between the template 40 and the base 62 of the clamping device 60.

FIG. 9 is a cross-sectional view of a portion of the extraction template 40 mounted to a portion of the multi-well plate 20 (the adhesive 24 is not shown in FIG. 9). In this configuration, the skirt 45b of the septa 44 masks or covers the side of the well 22 while leaving the majority of the well 22 bottom exposed for extraction of the biofilm 52 retained on the surface of the coating 50. The actual area of each coating 50 exposed for analysis, when the extraction template 40 is in place, is less than without the template (e.g., approximately 1.227 cm², as compared to 1.766 cm²). As a result, both the uncoated side wall of the well 22 and the outer periphery of the coating 50 (that may have been contaminated with the polystyrene) are excluded from the analysis. The water tight seal between the template 40 and the multi-well plate 20 prevents the the extraction solution 54 from leaking underneath the septa skirt 45b and eluting the crystal violet retained within the biofilm 52 on the side of the well 22 during the extraction procedure.

Due to the drying and staining procedures, bacterial films remain fixed to the sides of the well 22 during application of the extraction template 40 and can be visualized against the white background of each septa 44. The extraction solution 54, for example, 500 μL of 33% glacial acetic acid, is then added through the septa opening 45a and allowed to sit for about 10 minutes with occasional shaking to extract the crystal violet from the surface of the coating material 50. 150 μL aliquots of the eluate are then transferred to clean plates, (e.g., 96-well microtiter plates) for absorbance measurements at 600 nm with a multi-well plate reader. The extraction template 40 can be cleaned via immersion in methanol for 5 minutes to remove any residual crystal violet adsorbed onto septa 44.

### EXAMPLES

The following examples demonstrate possible use of the modified multi-well plates according to the present invention. Modified multi-well plates were utilized to assess the initial settlement/biofilm formation obtained on 4 different types of external reference coatings routinely utilized as controls for assessing antifouling/foul-release properties. Three silicone based resins (Dow Corning 3140, GE RTV-11 and GE T2-silastic) and an acrylate (Paraloid B-44S 40%, PMM) were deposited into a 24-well, glass coverslip/epoxy anchored modified polystyrene plate. The resins were allowed to cure for >=24 hours at room temperature and were subsequently pre-leached in deionized water for >=24 hours to remove any residual curing agent or solvent prior to analysis.

Three different marine bacteria were utilized to evaluate the initial settlement/biofilm formation on each coating type (Halomonas marina,Pseudoalteromonas atlantica, Vibrio anguillarum). Coatings were challenged with each bacterium individually for about 18 hours at 28° C. Methodologies adapted from the current literature were employed to carry out the quantification of biofilm obtained. See S. Stepanovic, D. Vukovic, I. Dakic, B. Savic, M. Svabic-Vlahovic. 2000. “A modified microtiter-plate test for quantification of staphylococcal biofilm formation”. J. OF MICROBIOL. METHODS. 40:175-179; D. Djordjevic, M. Wiedmann, and L. A. McLandsborough. 2002. “Microtiter Plate Assay for Assessment of Listeria monocytogenes Biofilm Formation”. APPLIED AND ENVIRON. MICROBIOL. 68(6):2950-2958. Briefly, planktonic and loosely adherent bacteria were removed from each well containing one of the external reference coatings by rinsing twice (2×) with 1.0 mL nanopure water. The remaining, adherent biofilm was immediately stained with 0.5 mL of 1% weight by volume (w/v) crystal violet solution for about 15 minutes. Excess stain was removed by rinsing three times (3×) with 1.0 mL of nanopure water. Plates were then inverted and firmly tapped several times against a paper towel to ensure that all remaining, non-bound stain was removed from each well. Plates were then allowed to dry at room temperature for about 1 hour or until visibly dry. 0.5 mL of a 33% by volume (v/v) glacial acetic acid/nanopure water solution was then added to each well for about 10 minutes (with gentle shaking) to elute the bound stain. 0.15 mL of the crystal violet/acetic acid solution was then transferred to a 96-well plate and measured for optical density (OD₆₀₀) with a standard multiwell plate reader.

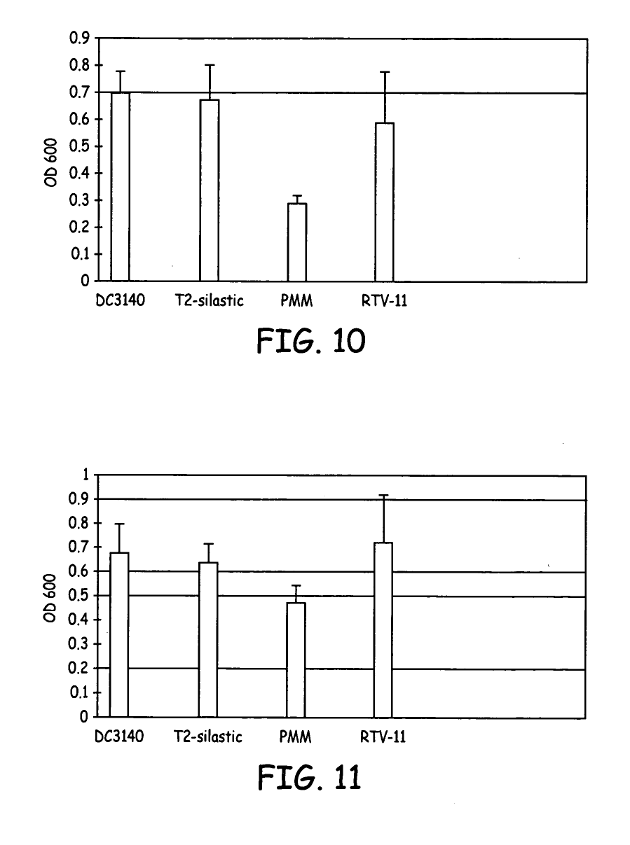

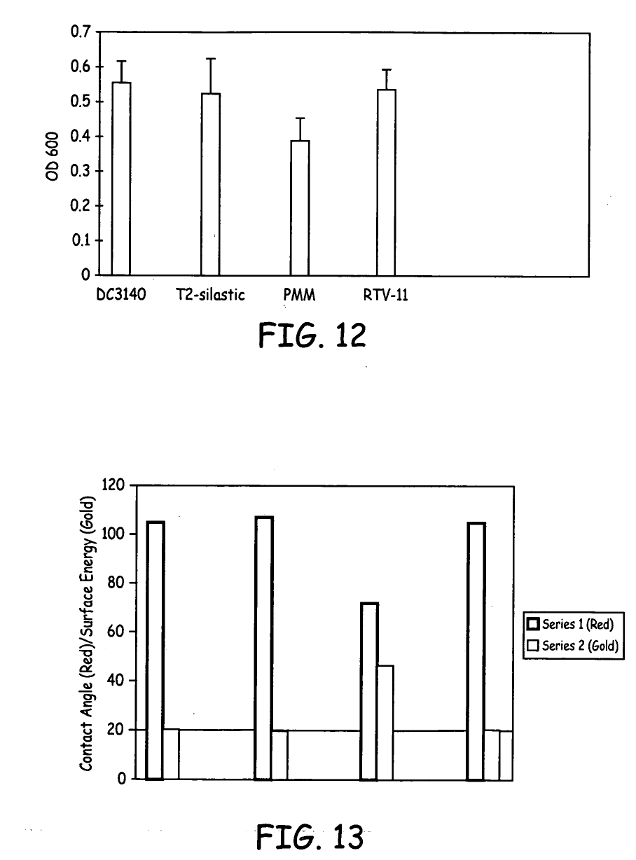

The cationic dye crystal violet (cv) is a standard biomass indicator wherein the OD₆₀₀ measurement is directly proportional to the biomass obtained on the surface of each coating. Therefore, a comparison of the OD₆₀₀ readings obtained for each coating type is utilized to evaluate their ability to inhibit settlement and initial biofilm growth. This information enables one to successfully identify superior performing candidates that warrant further testing and characterization. FIGS. 10, 11 and 12 are graphs showing that the results from each of the bacteria utilized are similar. This was anticipated from investigating the current literature as the marine bacteria utilized have a tendency to settle on more hydrophobic or lower surface energy surfaces. See L. K. Ista, V. H. Perez-Luna, and G. P. Lopez. 1999. “Surface-Grafted, 15 Environmentally Sensitive Polymers for Biofilm Release”. APPLIED AND ENVIRON. MICROBIOL. 65(4):1603-1609. As shown in the graph of FIG. 13, static contact angle measurements (series 1) and surface free energy (series 2) calculations with deionized water as the contact solvent were made for each of the four coating types.

Thus, the foregoing examples demonstrate how high-throughput testing and screening can be accomplished utilizing the modified multi-well plates of the present invention. More particularly, the foregoing examples demonstrate how biofilms can be used to test coating samples disposed on liners of multi-well plates according to the present invention.

Although the present invention has been described with reference to preferred embodiments, workers skilled in the art will recognize that changes maybe made in form and detail without departing from the spirit and scope of the invention.

1. A multi-well plate comprising:
a plurality of wells, each well having a base and one or more side walls; and
a liner disposed adjacent the base of at least one of the plurality of wells.

2. The multi-well plate of claim 1 wherein a liner is disposed adjacent the base of each of the plurality of wells.

3. The multi-well plate of claim 1 wherein the multi-well plate comprises a 24 well plate formed of polystyrene.

4. The multi-well plate of claim 3 wherein the liner comprises glass.

5. The multi-well plate of claim 4 wherein the glass liner comprises at least a portion of a microscope slip.

6. The multi-well plate of claim 1 wherein the liner is attached to the bottom surface of the well using an adhesive.

7. The multi-well plate of claim 6 wherein the adhesive comprises a two-part silicone adhesive.

8. The multi-well plate of claim 1 where the liner is attached to the multi-well plate using an adhesive-free mechanical attachment.

9. A method for forming a multi-well plate system, the method comprising:
providing a multi-well plate; and
applying a liner to a well of the multi-well plate.

10. The method of claim 9 and further comprising:
applying an adhesive to the well prior to applying the liner to adhere the liner to a surface of the well.

11. The method of claim 9 and further comprising:
providing an extraction template for removing materials from the liner.

12. The method of claim 11 and further comprising:
affixing the liner to te well using an adhesive-free mechanical connection.

13. A multi-well plate system comprising:
a plate having a plurality of wells; and
a liner affixed at a bottom portion of at least one of the plurality of wells, wherein the liner comprises a different material than at least the bottom portion of the at least one wells.

14. The system of claim 13 and further comprising an extraction template positionable on the plate.

15. The system of claim 14, wherein the extraction template comprises a block having a plurality of openings that correspond to the wells of the plate, such that the openings of the block and the wells of the plate can be generally aligned.

16. The system of claim 14, wherein the extraction template comprises a plurality of septa for at least partial insertion into one or more of the wells of the plate.

17. The system of claim 14 and further comprising a clamping device for securing the extraction template to the plate.

18. The system of claim 14, wherein the liner comprises a generally non-reactive material.

19. The system of claim 13, wherein the liner is affixed to the bottom portion of the at least one well with an adhesive.

20. The system of claim 19, wherein the adhesive is substantially optically transparent when cured.
